(19) United States
(12) Patent Application Publication
(10) Pub. No.: US 2024/0298937 A1
(43) Pub. Date: Sep. 12, 2024

(54) DETECTION DEVICE

(71) Applicant: Japan Display Inc., Tokyo (JP)

(72) Inventors: Atsunori OYAMA, Tokyo (JP); Kento HIMOTO, Tokyo (JP)

(21) Appl. No.: 18/593,127

(22) Filed: Mar. 1, 2024

(30) Foreign Application Priority Data
Mar. 8, 2023 (JP) 2023-035793

(51) Int. Cl. A61B 5/1455; A61B 5/00

(52) U.S. Cl. CPC A61B 5/14552; A61B 5/6802; A61B 2562/0233; A61B 2562/046

(57) ABSTRACT

According to an aspect, a detection device includes: a housing; an optical sensor including photodiodes; a light source disposed in the housing so as to be close to an end of the optical sensor; and a control circuit configured to control light emission of the light source. The optical sensor includes gate lines arranged in a first direction away from the light source and electrically coupled to the photodiodes. The control circuit is configured to control the light emission of the light source such that, when the photodiodes are sequentially driven on a gate line basis, a total amount of light of the light source when detected by the photodiodes driven by the gate line closest to the light source is smaller than the total amount of light of the light source when detected by the photodiodes driven by the gate line farthest from the light source.

### CROSS-REFERENCE TO RELATED APPLICATION

This application claims the benefit of priority from Japanese Patent Application No. 2023-035793 filed on Mar. 8, 2023, the entire contents of which are incorporated herein by reference.

### BACKGROUND

### 1. Technical Field

What is disclosed herein relates to a detection device.

### 2. Description of the Related Art

Devices are known that detect information on a living body from a human body. Japanese Patent Application Laid-open Publication No. 2012-065900 discloses a pulse wave sensor that can measure pulse waves without constraining the action of a test subject.

A photodiode can produce an appropriate output within a range (measurement range) of intensity of light of a light source. When a planar optical sensor including a plurality of photodiodes and a light source are adjacently arranged in a housing, the photodiodes closer to the light source may not be able to make measurements because they are illuminated at excessively high brightness levels that exceed the measurement range, and the photodiodes farther from the light source may not be able to make measurements because they are illuminated at excessively low brightness levels that do not reach the measurement range.

For the foregoing reasons, there is a need for a detection device that allows a planar optical sensor and a light source to be arranged close to each other.

### SUMMARY

According to an aspect, a detection device includes: a housing; an optical sensor that includes a plurality of photodiodes provided in a matrix having a row-column configuration and is provided in a planar configuration in the housing; a light source disposed in the housing so as to be close to an end of the optical sensor; and a control circuit configured to control light emission of the light source. The optical sensor includes a plurality of gate lines arranged in a first direction away from the light source and electrically coupled to the photodiodes. The control circuit is configured to control the light emission of the light source such that, when the photodiodes are sequentially driven on a gate line basis, a total amount of light of the light source when detected by the photodiodes driven by the gate line closest to the light source is smaller than the total amount of light of the light source when detected by the photodiodes driven by the gate line farthest from the light source.

### BRIEF DESCRIPTION OF THE DRAWINGS

FIG. 1 is a schematic view illustrating an example of an external view of a state where a finger is accommodated in a detection device according to an embodiment of the present disclosure, as viewed from a lateral side of a housing;

FIG. 2 is a sectional view along section II-II′ illustrated in FIG. 1;

FIG. 3 is a sectional view along section III-III′ illustrated in FIG. 2;

FIG. 4 is a schematic view obtained by developing a light source and an optical sensor illustrated in FIG. 2;

FIG. 5 is a configuration diagram illustrating an example of the light source and the optical sensor of the detection device according to the embodiment;

FIG. 6 is a block diagram illustrating a configuration example of the detection device according to the embodiment;

FIG. 7 is a circuit diagram illustrating a circuit configuration example of the detection device;

FIG. 8 is a circuit diagram illustrating a plurality of partial detection areas;

FIG. 9 is a schematic partial sectional view of the optical sensor of the embodiment;

FIG. 10 is a diagram illustrating exemplary relations between a distance and a light intensity of the optical sensor illustrated in FIG. 4;

FIG. 11 is a diagram for explaining a control example of a control circuit;

FIG. 12 is a timing waveform diagram illustrating an example of first control of the detection device;

FIG. 13 is a timing waveform diagram illustrating an example of second control of the detection device;

FIG. 14 is a timing waveform diagram illustrating an example of third control of the detection device;

FIG. 15 is a timing waveform diagram illustrating an example of fourth control of the detection device; and

FIG. 16 is a schematic view obtained by developing another configuration example of the light source of the detection device according to the embodiment.

### DETAILED DESCRIPTION

The following describes modes (embodiments) for carrying out the present invention in detail with reference to the drawings. The present disclosure is not limited to the description of the embodiments given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the invention. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the description and the drawings, and detailed description thereof may not be repeated where appropriate.

In the present disclosure, in expressing an aspect of disposing another structure on or above a certain structure, a case of simply expressing “on” includes both a case of disposing the other structure immediately on the certain structure so as to contact the certain structure and a case of disposing the other structure above the certain structure with still another structure interposed therebetween, unless otherwise specified.

### Embodiment

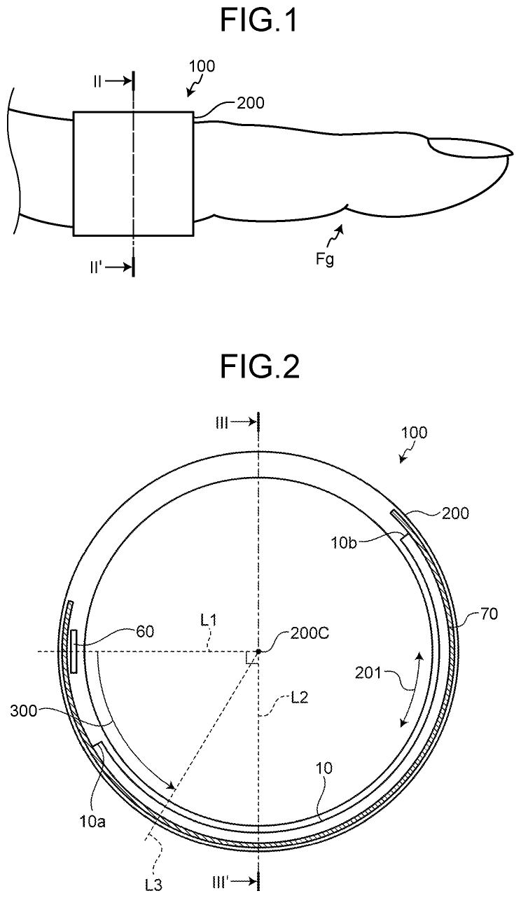

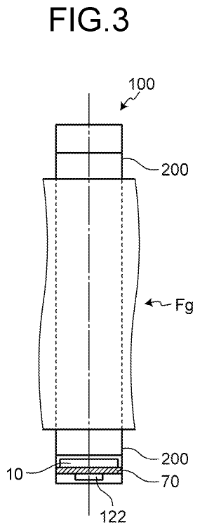

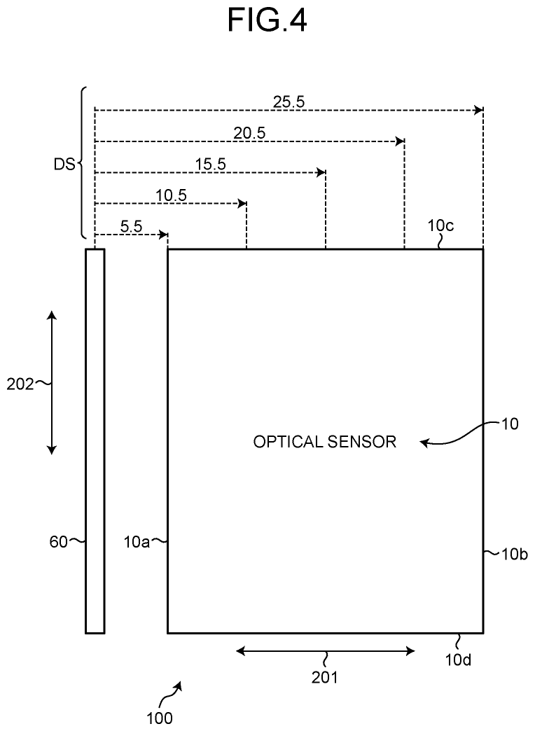

FIG. 1 is a schematic view illustrating an example of an external view of a state where a finger, thumb, or toe (hereinafter, collectively referred to as a finger) is accommodated in a detection device according to an embodiment of the present disclosure, as viewed from a lateral side of a housing. FIG. 2 is a sectional view along section II-II′ illustrated in FIG. 1. FIG. 3 is a sectional view along section III-III′ illustrated in FIG. 2. FIG. 4 is a schematic view obtained by developing a light source and an optical sensor illustrated in FIG. 2.

A detection device 100 illustrated in FIGS. 1 and 2 is a finger ring-shaped device that can be worn on and removed from a human body, and is worn on an object to be detected Fg. The object to be detected Fg of the embodiment is a finger, and may be any one of a thumb, an index finger, a middle finger, a ring finger, and a little finger. The detection device 100 can detect biometric information on a living body from the object to be detected Fg on which the detection device 100 is worn.

As illustrated in FIG. 2, the detection device 100 includes at least a housing 200, and a light source 60 and an optical sensor 10 provided on a flexible substrate 70. The housing 200 accommodates therein the light source 60 and the optical sensor 10. FIG. 2 does not illustrate components other than the housing 200, the substrate 70, the light source 60, and the optical sensor 10. The detection device 100 operates on power from a battery, which is not illustrated.

The housing 200 is formed in a ring shape (annular shape) that can be worn on the object to be detected Fg, and is a mounting member worn on the living body. The housing 200 is formed of a housing material such as a synthetic resin. The outer surface of the housing 200 is a light-blocking resin. As a result, noise by external light is reduced. The inner surface of the housing 200 is a light-transmitting resin. As a result, light emitted by the light source 60 can be applied to the object to be detected Fg, and light from the object to be detected Fg can be received by the optical sensor 10.

In the embodiment, the detection device 100 will be described as having a configuration including the housing 200, the substrate 70, the light source 60, and the optical sensor 10. The detection device 100 may, however, have a configuration including the housing 200, the light source 60, and the optical sensor 10.

As illustrated in FIGS. 2 and 3, in the detection device 100, the optical sensor 10 is located inside the substrate 70 so that the optical sensor 10 can receive the light from the object to be detected Fg. The detection device 100 accommodates the light source 60 and the optical sensor 10 in the housing 200 such that one end 10a of the optical sensor 10 is closer to the light source 60 and another end 10b of the optical sensor 10 is farther from the light source 60 in a peripheral direction 201 of the housing 200. As illustrated in FIG. 3, in the detection device 100, a control circuit 122 is disposed on the outside of the substrate 70.

As illustrated in FIG. 4, the one end 10a and the other end 10b of the optical sensor 10 are the long sides of the substantially rectangular shaped optical sensor 10 that intersect in the peripheral direction 201 of the housing 200 and extend along a width direction 202 of the housing 200. A side 10c and a side 10d of the optical sensor 10 are the short sides of the optical sensor 10 that extend along the peripheral direction 201. On the substrate 70, the light source 60 and the optical sensor 10 are mounted such that the one end 10a of the optical sensor 10 is close to the light source 60 in the peripheral direction 201. In the detection device 100, the substrate 70 is accommodated in the housing 200, so that the light source 60 and the optical sensor 10 are disposed in the housing 200 while being close to each other.

In the example illustrated in FIG. 4, the detection device 100 has a distance DS of 5.5 mm from the light source 60 to the one end 10a of the optical sensor 10 and the distance DS of 25.5 mm from the light source 60 to the other end 10b of the optical sensor 10. The distances DS represent distances from the light source 60 to a plurality of locations of the optical sensor 10 in the housing 200.

For example, inorganic light-emitting diodes (LEDs) or organic electroluminescent (EL) diodes (organic light-emitting diodes (OLEDs)) are used as the light source 60, as illustrated in FIGS. 2 and 4. The light source 60 emits light having predetermined wavelengths. In the present embodiment, the light source 60 includes a first light-emitting element that emits red color and a second light-emitting element that emits near-infrared light. First light has a wavelength of approximately 660 nm, for example, and second light has a wavelength of approximately 850 nm, for example.

The detection device 100 has a function to detect a blood oxygen level in addition to pulse waves, pulsation, and a vascular image as the information on a living body based on the first light and the second light emitted from the light source 60. Red blood cells contained in blood contain hemoglobin. The near-infrared light emitted from the light source 60 can be easily absorbed by hemoglobin. In other words, the coefficient of absorption of the near-infrared light by hemoglobin is higher than that of the other parts in the body. Therefore, the optical sensor 10 can detect a vascular pattern of veins or the like by reading the amount of light received by a plurality of photodiodes and identifying areas where the amount of near-infrared light received is relatively low.

Reflected light of near-infrared and red light contains information for measuring oxygen saturation in the blood (hereinafter, referred to as a “blood oxygen saturation level (SpO₂)”). The blood oxygen saturation level (SpO₂) refers to a ratio of an amount of oxygen actually bound to hemoglobin to the total amount of oxygen under the assumption that the oxygen is bound to all the hemoglobin in the blood. When calculating the blood oxygen saturation level (SpO₂), a pulse wave acquired using the first light and a pulse wave acquired using the second light are used.

Hemoglobin in blood contains oxygenated hemoglobin and reduced hemoglobin. The blood oxygen saturation level (SpO₂) is determined by the ratio of hemoglobin in blood bound to oxygen (oxygenated hemoglobin (O₂Hb)) to hemoglobin in blood not bound to oxygen (reduced hemoglobin (HHb)).

The light absorption characteristics of the red light are represented as HHb>>O₂Hb, indicating that HHb has significantly larger absorbance, while the light absorption characteristics of the near-infrared light are represented as HHb≈O₂Hb, indicating that O₂Hb has slightly larger absorbance.

Using this difference in light absorption characteristics, the blood oxygen saturation level (SpO₂) can be evaluated using the ratio of the measurement value of the near-infrared light to the measurement value of the red light.

In the present disclosure, the light emitted from the light source 60 is not limited to the above-described types of light. The light source 60 may emit only near-infrared light having a wavelength of 800 nm or larger and smaller than 1000 nm, or only red light having a wavelength of 600 nm or larger and smaller than 800 nm, depending on the application.

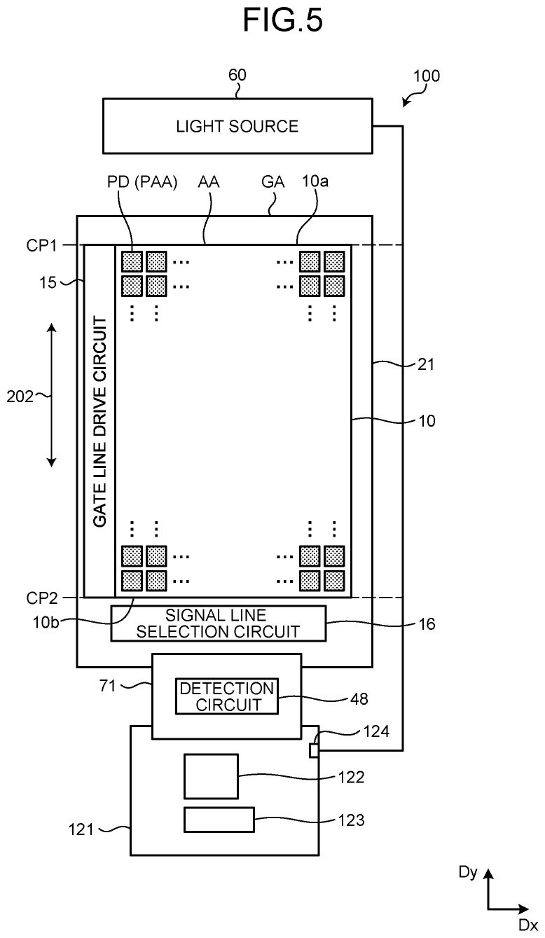

FIG. 5 is a configuration diagram illustrating an example of the light source 60 and the optical sensor 10 of the detection device 100 according to the embodiment. In the example illustrated in FIG. 5, the optical sensor 10 includes a sensor substrate 21.

The sensor substrate 21 is electrically coupled to a control substrate 121 through a flexible printed circuit board 71. The flexible printed circuit board 71 is provided with a detection circuit 48. The control substrate 121 is provided with the control circuit 122 and a power supply circuit 123. The control circuit 122 is, for example, a field-programmable gate array (FPGA). The control circuit 122 supplies control signals to the optical sensor 10, a gate line drive circuit 15, and a signal line selection circuit 16 to control a detection operation of the optical sensor 10. The control circuit 122 also supplies control signals to the light source 60 to control lighting and non-lighting of the light source 60. The power supply circuit 123 supplies voltage signals including, for example, a sensor power supply signal (sensor power supply voltage) VDDSNS to the optical sensor 10, the gate line drive circuit 15, and the signal line selection circuit 16. The power supply circuit 123 also supplies a power supply voltage to the light source 60.

The sensor substrate 21 has a detection area AA and a peripheral area GA. The detection area AA is an area provided with a plurality of photodiodes PD included in the optical sensor 10. The peripheral area GA is an area between the outer perimeter of the detection area AA and the edges of the sensor substrate 21, and is an area not overlapping the photodiodes PD.

One side CP1 of the four sides of the detection area AA forming a boundary between the rectangular detection area AA and the peripheral area GA is one end of a first area 230 and is the one end 10a of the optical sensor 10. Another side CP2 of the four sides of the detection area AA that is located opposite the one side with the detection area AA interposed therebetween is the other end of the first area 230 and is the other end 10b of the optical sensor 10.

The gate line drive circuit 15 and the signal line selection circuit 16 are provided in the peripheral area GA. Specifically, the gate line drive circuit 15 is provided in an area extending along a second direction Dy in the peripheral area GA. The signal line selection circuit 16 is provided in an area extending along a first direction Dx in the peripheral area GA, and is provided between the optical sensor 10 and the detection circuit 48.

The first direction Dx is one direction in a plane parallel to the sensor substrate 21. The second direction Dy is one direction in the plane parallel to the sensor substrate 21, and is a direction orthogonal to the first direction Dx. The second direction Dy may non-orthogonally intersect the first direction Dx. In the present embodiment, the first direction Dx is the width direction of the housing 200 and a direction along the one end 10a of the optical sensor 10. The second direction Dy is the peripheral direction 201 of the housing 200.

The light source 60 is mounted on a light source base material, which is electrically coupled to the control circuit 122 and the power supply circuit 123 via a terminal 124. The light source 60 is configured to be switchable between emitting the near-infrared light having a wavelength of 880 nm and emitting the red light having a wavelength of 665 nm under the control of the control circuit 122.

In the present disclosure, the wavelengths of light emitted by the light source 60 are not limited to the wavelengths mentioned above. The light source 60 only needs to be capable of emitting the near-infrared light having a wavelength of 800 nm or larger and smaller than 1000 nm as the first light. The light source 60 only needs to be capable of emitting the red light having a wavelength of 600 nm or larger and smaller than 800 nm as the second light.

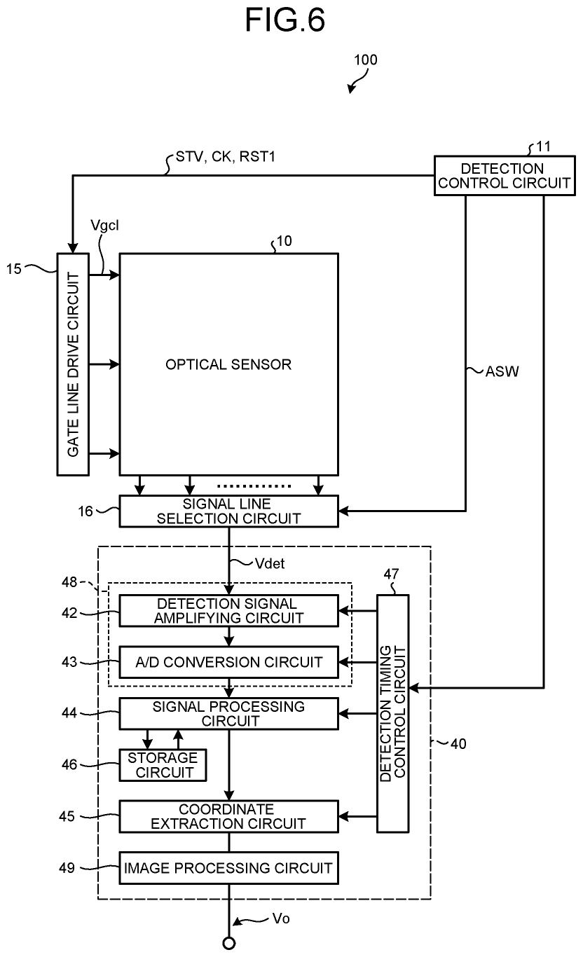

FIG. 6 is a block diagram illustrating a configuration example of the detection device 100 according to the embodiment. As illustrated in FIG. 6, the detection device 100 further includes a detection control circuit 11 and a detector (detection processing circuitry) 40. The control circuit 122 includes one, some, or all functions of the detection control circuit 11. The control circuit 122 also includes one, some, or all functions of the detector 40 other than those of the detection circuit 48.

The optical sensor 10 is an optical sensor that includes the photodiodes PD serving as photoelectric conversion elements. Each of the photodiodes PD included in the optical sensor 10 outputs an electrical signal corresponding to light applied to the photodiode PD as a detection signal Vdet to the signal line selection circuit 16. The optical sensor 10 performs the detection in response to a gate drive signal Vgcl supplied from the gate line drive circuit 15.

The detection control circuit 11 is a circuit that supplies respective control signals to the gate line drive circuit 15, the signal line selection circuit 16, and the detector 40 to control operations of these components. The detection control circuit 11 supplies various control signals including, for example, a start signal STV, a clock signal CK, and a reset signal RST1 to the gate line drive circuit 15. The detection control circuit 11 also supplies various control signals including, for example, a selection signal ASW to the signal line selection circuit 16. The detection control circuit 11 also supplies various control signals to the light source 60 to control the lighting and non-lighting of light source 60.

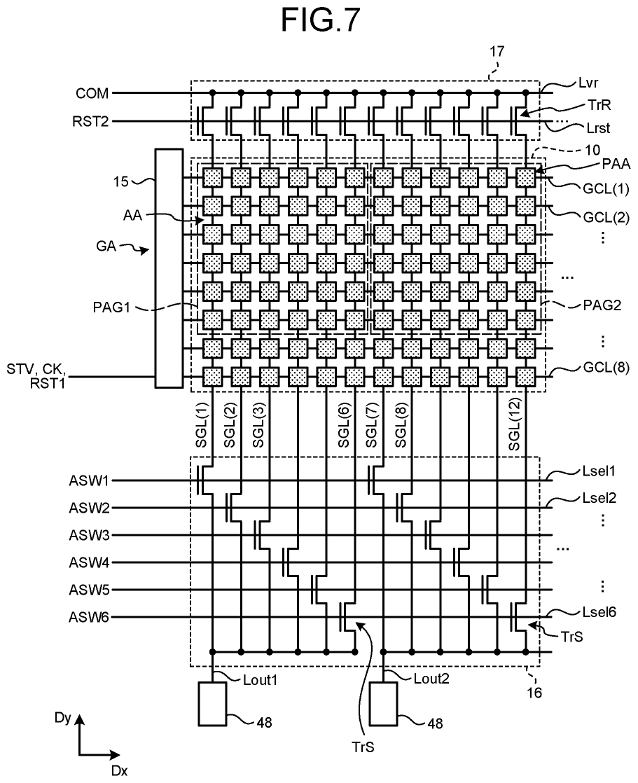

The gate line drive circuit 15 is a circuit that drives a plurality of gate lines GCL (refer to FIG. 7) based on the various control signals. The gate line drive circuit 15 sequentially or simultaneously selects the gate lines GCL, and supplies the gate drive signals Vgcl to the selected gate lines GCL. By this operation, the gate line drive circuit 15 selects the photodiode PD coupled to the gate lines GCL.

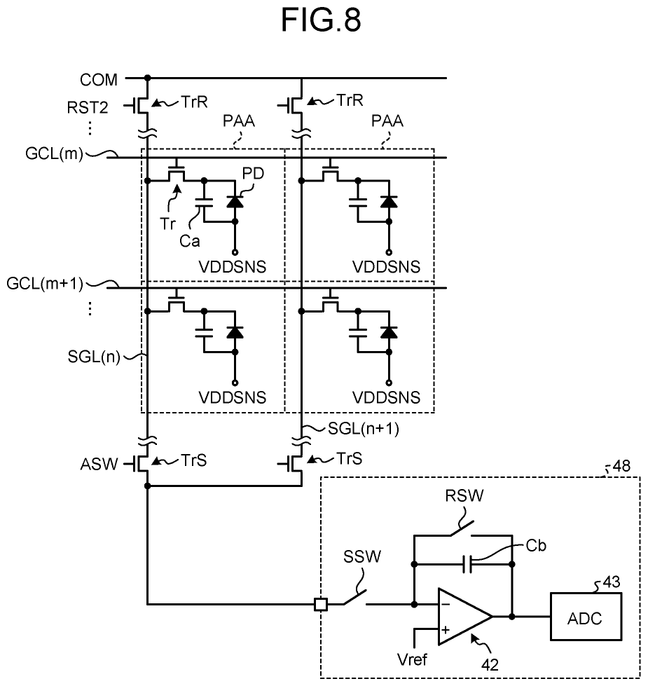

The signal line selection circuit 16 is a switch circuit that sequentially or simultaneously selects a plurality of signal lines SGL (refer to FIG. 8). The signal line selection circuit 16 is, for example, a multiplexer. The signal line selection circuit 16 couples the selected signal lines SGL to the detection circuit 48 based on the selection signal ASW supplied from the detection control circuit 11. By this operation, the signal line selection circuit 16 outputs the detection signals Vdet of the photodiodes PD to the detector 40.

The detector 40 includes the detection circuit 48, a signal processing circuit 44, a coordinate extraction circuit 45, a storage circuit 46, a detection timing control circuit 47, and an image processing circuit 49. The detection timing control circuit 47 performs control to cause the detection circuit 48, the signal processing circuit 44, the coordinate extraction circuit 45, and the image processing circuit 49 such that they operate in synchronization with one another based on a control signal supplied from the detection control circuit 11.

The detection circuit 48 is, for example, an analog front-end (AFE) circuit. The detection circuit 48 is a signal processing circuit having functions of at least a detection signal amplifying circuit 42 and an analog-to-digital (A/D) conversion circuit 43. The detection signal amplifying circuit 42 amplifies the detection signal Vdet. The A/D conversion circuit 43 converts an analog signal output from the detection signal amplifying circuit 42 into a digital signal.

The signal processing circuit 44 is a logic circuit that detects a predetermined physical quantity received by the optical sensor 10 based on output signals of the detection circuit 48. The signal processing circuit 44 can detect asperities on a biological surface of the object to be detected Fg or a palm based on the signals from the detection circuit 48 when the object to be detected Fg is in contact with or in proximity to a detection surface. The signal processing circuit 44 can detect the information on the living body based on the signals from the detection circuit 48. Examples of the information on the living body include the pulsation, the blood oxygen saturation level, and the like of the object to be detected Fg.

The signal processing circuit 44 may also perform processing of acquiring the detection signals Vdet (information on the living body) simultaneously detected by the photodiodes PD, and averaging the detection signals Vdet. In this case, the detector 40 can perform stable detection by reducing measurement errors caused by noise or relative positional misalignment between the object to be detected Fg and the optical sensor 10.

The storage circuit 46 temporarily stores therein signals calculated by the signal processing circuit 44. The storage circuit 46 may be, for example, a random-access memory (RAM) or a register circuit.

The coordinate extraction circuit 45 is a logic circuit that obtains detected coordinates of the asperities on the biological surface of the finger or the like when the contact or proximity of the finger is detected by the signal processing circuit 44. The coordinate extraction circuit 45 is the logic circuit that also obtains detected coordinates of blood vessels in the object to be detected Fg. The image processing circuit 49 combines the detection signals Vdet output from the photodiodes PD of the optical sensor 10 to generate two-dimensional information representing the shape of the asperities on the biological surface of the object to be detected Fg or the like and two-dimensional information representing the shape of the blood vessels in the object to be detected Fg. The coordinate extraction circuit 45 may output the detection signals Vdet as sensor output Vo instead of calculating the detected coordinates. A case can be considered where the detector 40 does not include the coordinate extraction circuit 45 and the image processing circuit 49.

The detection control circuit 11 has a function to compare the detected information on the living body with authentication information stored in advance and authenticate a person to be authenticated based on the result of the comparison. The detection control circuit 11 has a function to control transmission of the detected information on the living body to an external device through a communication device, which is not illustrated in the drawings.

The following describes a circuit configuration example of the detection device 100. FIG. 7 is a circuit diagram illustrating a circuit configuration example of the detection device 100. FIG. 8 is a circuit diagram illustrating the partial detection areas. FIG. 8 also illustrates a circuit configuration of the detection circuit 48.

As illustrated in FIG. 7, the optical sensor 10 has a plurality of partial detection areas PAA arranged in a matrix having a row-column configuration. Each of the partial detection areas PAA is provided with the photodiode PD.

The gate lines GCL extend in the first direction Dx, and are each coupled to the partial detection areas PAA arranged in the first direction Dx. A plurality of gate lines GCL(1), GCL(2), . . . , GCL(8) are arranged in the second direction Dy, and are each coupled to the gate line drive circuit 15. In the following description, the gate lines GCL(1), GCL(2), . . . , GCL(8) will each be simply referred to as the gate line GCL when they need not be distinguished from one another. For ease of understanding of the description, FIG. 7 illustrates eight of the gate lines GCL. However, this is merely an example, and M gate lines GCL may be arranged (where M is 8 or larger, and is, for example, 256).

The signal lines SGL extend in the second direction Dy and are each coupled to the photodiodes PD in the partial detection areas PAA arranged in the second direction Dy. A plurality of signal lines SGL(1), SGL(2), . . . , SGL(12) are arranged in the first direction Dx and are each coupled to the signal line selection circuit 16 and a reset circuit 17. In the following description, the signal lines SGL(1), SGL(2), . . . , SGL(12) will each be simply referred to as the signal line SGL when they need not be distinguished from one another.

For ease of understanding of the description, 12 of the signal lines SGL are illustrated. However, this is merely an example, and N signal lines SGL may be arranged (where N is 12 or larger, and is, for example, 252). In FIG. 7, the optical sensor 10 is provided between the signal line selection circuit 16 and the reset circuit 17. The signal line selection circuit 16 and the reset circuit 17 are not limited to being provided in this manner, but may be coupled to ends of the signal lines SGL on the same side.

The gate line drive circuit 15 receives the various control signals such as the start signal STV, the clock signal CK, and the reset signal RST1 from the control circuit 122 (refer to FIG. 5). The gate line drive circuit 15 sequentially selects the gate lines GCL(1), GCL(2), . . . , GCL(8) in a time-division manner based on the various control signals. The gate line drive circuit 15 supplies the gate drive signal Vgcl to the selected one of the gate lines GCL. This operation supplies the gate drive signal Vgcl to a plurality of first switching elements Tr coupled to the gate line GCL, and the partial detection areas PAA arranged in the first direction Dx are selected as detection targets.

The gate line drive circuit 15 may perform different driving for each of detection modes including the detection of a fingerprint or a thumbprint and the detection of different items of the information on the living body (such as the pulsation and the blood oxygen saturation level). For example, the gate line drive circuit 15 may collectively drive more than one of the gate lines GCL.

Specifically, the gate line drive circuit 15 simultaneously selects a predetermined number of the gate lines GCL from among the gate lines GCL(1), GCL(2), . . . , GCL(8) based on the control signals. For example, the gate line drive circuit 15 simultaneously selects six gate lines GCL(1) to GCL(6) and supplies thereto the gate drive signals Vgcl. The gate line drive circuit 15 supplies the gate drive signals Vgcl through the selected six gate lines GCL to the first switching elements Tr. By this operation, detection area groups PAG1 and PAG2 each including more than one of the partial detection areas PAA arranged in the first direction Dx and the second direction Dy are selected as the detection targets. The gate line drive circuit 15 collectively drives the predetermined number of the gate lines GCL, and sequentially supplies the gate drive signals Vgcl to the gate lines GCL for each unit of the predetermined number of the gate lines GCL.

The signal line selection circuit 16 includes a plurality of selection signal lines Lsel, a plurality of output signal lines Lout, and a plurality of third switching elements TrS. The third switching elements TrS are provided correspondingly to the signal lines SGL. Six signal lines SGL(1), SGL(2), . . . , SGL(6) are coupled to a common output signal line Lout1. Six signal lines SGL(7), SGL(8), . . . , SGL(12) are coupled to a common output signal line Lout2. The output signal lines Lout1 and Lout2 are each coupled to the detection circuit 48.

The signal lines SGL(1), SGL(2), . . . , SGL(6) are grouped into a first signal line block, and the signal lines SGL(7), SGL(8), . . . , SGL(12) are grouped into a second signal line block. The selection signal lines Lsel are coupled to the gates of the respective third switching elements TrS included in one of the signal line blocks. One of the selection signal lines Lsel is coupled to the gates of the third switching elements TrS in the signal line blocks.

Specifically, selection signal lines Lsel1, Lsel2, . . . , Lsel6 are coupled to the third switching elements TrS corresponding to the signal lines SGL(1), SGL(2), . . . , SGL(6), respectively. The selection signal line Lsel1 is coupled to one of the third switching elements TrS corresponding to the signal line SGL(1) and one of the third switching elements TrS corresponding to the signal line SGL(7). The selection signal line Lsel2 is coupled to one of the third switching elements TrS corresponding to the signal line SGL(2) and one of the third switching elements TrS corresponding to the signal line SGL(8).

The control circuit 122 (refer to FIG. 5) sequentially supplies the selection signal ASW to the selection signal lines Lsel. This operation causes the signal line selection circuit 16 to operate the third switching elements TrS to sequentially select the signal lines SGL in one of the signal line blocks in a time-division manner. The signal line selection circuit 16 selects one of the signal lines SGL in each of the signal line blocks. With the above-described configuration, the detection device 100 can reduce the number of integrated circuits (ICs) including the detection circuit 48 or the number of terminals of the ICs.

The signal line selection circuit 16 may collectively couple more than one of the signal lines SGL to the detection circuit 48. Specifically, the control circuit 122 (refer to FIG. 5) simultaneously supplies the selection signal ASW to the selection signal lines Lsel. This operation causes the signal line selection circuit 16 to operate the third switching elements TrS to select the signal lines SGL (for example, six of the signal lines SGL) in one of the signal line blocks, and couple the signal lines SGL to the detection circuit 48. As a result, signals detected in each of the detection area groups PAG1 and PAG2 are output to the detection circuit 48. In this case, the signals from the partial detection areas PAA (photodiodes PD) included in each of the detection area groups PAG1 and PAG2 are integrated and output to the detection circuit 48.

By the operations of the gate line drive circuit 15 and the signal line selection circuit 16, the detection is performed for each of the detection area groups PAG1 and PAG2. As a result, the intensity of the detection signal Vdet obtained by a one-time detection operation increases, so that the sensor sensitivity can be improved. In addition, time required for the detection can be reduced. As a result, the detection device 100 can repeatedly perform the detection in a short time, and thus, can improve a signal-to-noise (S/N) ratio, and can accurately detect a change over time of the information on the living body, such as a pulse wave.

As illustrated in FIG. 7, the reset circuit 17 includes a reference signal line Lvr, a reset signal line Lrst, and fourth switching elements TrR. The fourth switching elements TrR are provided correspondingly to the signal lines SGL. The reference signal line Lvr is coupled to either the sources or the drains of the fourth switching elements TrR. The reset signal line Lrst is coupled to the gates of the fourth switching elements TrR.

The control circuit 122 supplies a reset signal RST2 to the reset signal line Lrst. This operation turns on the fourth switching elements TrR to electrically couple the signal lines SGL to the reference signal line Lvr. The power supply circuit 123 supplies a reference signal COM to the reference signal line Lvr. This operation supplies the reference signal COM to a capacitive element Ca (refer to FIG. 8) included in each of the partial detection areas PAA.

As illustrated in FIG. 8, each of the partial detection areas PAA includes the photodiode PD, the capacitive element Ca, and the first switching element Tr. FIG. 8 illustrates two gate lines GCL(m) and GCL(m+1) arranged in the second direction Dy among the gate lines GCL. FIG. 8 also illustrates two signal lines SGL(n) and SGL(n+1) arranged in the first direction Dx among the signal lines SGL. The partial detection area PAA is an area surrounded by the gate lines GCL and the signal lines SGL. The first switching elements Tr are provided correspondingly to the photodiodes PD. The first switching element Tr is formed of a thin-film transistor, and in this example, formed of an n-channel metal oxide semiconductor (MOS) thin-film transistor (TFT).

The gates of the first switching elements Tr belonging to the partial detection areas PAA arranged in the first direction Dx are coupled to the gate line GCL. The sources of the first switching elements Tr belonging to the partial detection areas PAA arranged in the second direction Dy are coupled to the signal line SGL. The drain of the first switching element Tr is coupled to the cathode of the photodiode PD and the capacitive element Ca.

The anode of the photodiode PD is supplied with the sensor power supply signal VDDSNS from the power supply circuit 123. The signal line SGL and the capacitive element Ca are supplied with the reference signal COM that serves as an initial potential of the signal line SGL and the capacitive element Ca from the power supply circuit 123.

When the partial detection area PAA is irradiated with light, a current corresponding to the amount of the light flows through the photodiode PD. As a result, an electric charge is stored in the capacitive element Ca. After the first switching element Tr is turned on, a current corresponding to the electric charge stored in the capacitive element Ca flows through the signal line SGL. The signal line SGL is coupled to the detection circuit 48 through a corresponding one of the third switching elements TrS of the signal line selection circuit 16. Thus, the detection device 100 can detect a signal corresponding to the amount of the light applied to the photodiode PD in each of the partial detection areas PAA or signals corresponding to the amounts of the light applied to the photodiodes PD in each of the detection area groups PAG1 and PAG2.

During a read period, a switch SSW of the detection circuit 48 is turned on, and the detection circuit 48 is coupled to the signal lines SGL. The detection signal amplifying circuit 42 of the detection circuit 48 converts a current or an electric charge supplied from the signal line SGL into a voltage corresponding thereto. A reference voltage Vref having a fixed potential is supplied to a non-inverting input terminal (+) of the detection signal amplifying circuit 42, and the signal lines SGL are coupled to an inverting input terminal (−) of the detection signal amplifying circuit 42. In the present embodiment, the same signal as the reference signal COM is supplied as the reference voltage Vref. The detection signal amplifying circuit 42 includes a capacitive element Cb and a reset switch RSW. During a reset period, the reset switch RSW is turned on, and the electric charge of the capacitive element Cb is reset.

With the above-described configuration, the detection device 100 including the photodiodes PD can detect the information on the living body, such as a vein pattern, a dermatoglyphic pattern, the blood oxygen saturation level, and the pulsation of the object to be detected Fg, and externally supply the biometric information including the detected information.

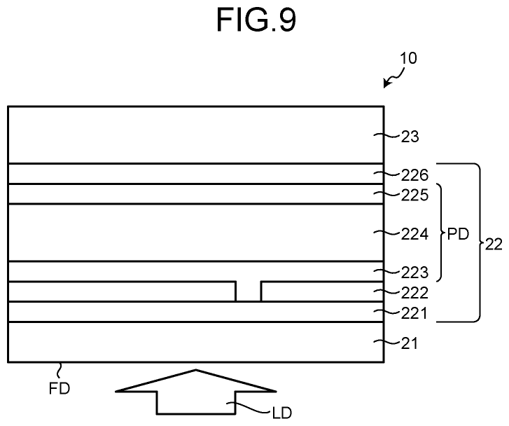

FIG. 9 is a schematic partial sectional view of the optical sensor of the embodiment. As illustrated in FIG. 9, the optical sensor 10 includes the sensor substrate 21, a sensor structure 22, and a protective film 23. The sensor substrate 21 is an insulating base material and is a second flexible substrate formed of a film-like resin.

The sensor structure 22 includes a TFT layer 221, a cathode electrode 222, the photodiode PD, and an anode electrode 226.

The TFT layer 221 is provided with TFTs, such as the first switching element Tr, and various types of wiring, such as the gate lines GCL and the signal lines SGL. The sensor substrate 21 and the TFT layer 221 serve as a drive circuit board that drives the sensor on a predetermined partial detection area PAA basis and are also called a backplane.

The photodiode PD includes an active layer 224, a hole transport layer 223 provided between the active layer 224 and the cathode electrode 222, and an electron transport layer 225 provided between the active layer 224 and the anode electrode 226. In other words, the hole transport layer 223, the active layer 224, and the electron transport layer 225 of the photodiode PD are stacked in this order in a direction orthogonal to the sensor substrate 21.

The active layer 224 changes in characteristics (for example, voltage-current characteristics and resistance value) according to light that illuminates the active layer 224. An organic material is used as a material of the active layer 224. Specifically, the active layer 224 has a bulk heterostructure containing a mixture of a p-type organic semiconductor and an n-type fullerene derivative (PCBM) that is an n-type organic semiconductor. As the active layer 224, low-molecular-weight organic materials can be used including, for example, fullerene (C₆₀), phenyl-C₆₁-butyric acid methyl ester (PCBM), copper phthalocyanine (CuPc), fluorinated copper phthalocyanine (F₁₆CuPc), 5,6,11,12-tetraphenyltetracene (rubrene), and perylene diimide (PDI) (derivative of perylene).

The active layer 224 can be formed by a vapor deposition process (dry process) using the low-molecular-weight organic materials listed above. In this case, the active layer 224 may be, for example, a multilayered film of CuPc and F₁₆CuPc, or a multilayered film of rubrene and C₆₀. The active layer 224 can also be formed by a coating process (wet process). In this case, the active layer 224 is made using a material obtained by combining the above-listed low-molecular-weight organic materials with a high-molecular-weight organic material. As the high-molecular-weight organic material, for example, poly(3-hexylthiophene) (P3HT) and F8-alt-benzothiadiazole (F8BT) can be used. The active layer 224 can be a film made of a mixture of P3HT and PCBM, or a film made of a mixture of F8BT and PDI.

The hole transport layer 223 and the electron transport layer 225 are provided to facilitate holes and electrons generated in the active layer 224 to reach the cathode electrode 222 and the anode electrode 226. The hole transport layer 223 is in direct contact with the top of the cathode electrode 222 through an opening OP of an insulating film 95. The active layer 224 is in direct contact with the top of the hole transport layer 223. The hole transport layer 223 is a metal-oxide layer. For example, tungsten oxide (WO₃) or molybdenum oxide is used as the oxide-metal layer.

The electron transport layer 225 is in direct contact with the top of the active layer 224, and the anode electrode 226 is in direct contact with the top of the electron transport layer 225. Polyethylenimine ethoxylated (PEIE) is used as a material of the electron transport layer 225.

The materials and the manufacturing methods of the hole transport layer 223, the active layer 224, and the electron transport layer 225 are merely exemplary, and other materials and manufacturing methods may be used.

The cathode electrode 222 faces the anode electrode 226 with the photodiode PD interposed therebetween. For example, a light-transmitting conductive material such as indium tin oxide (ITO) is used as the anode electrode 226. A metal material such as silver (Ag) or aluminum (Al) is used as the cathode electrode 222. Alternatively, the cathode electrode 222 may be made of an alloy material containing at least one or more of these metal materials.

By controlling the film thickness of the cathode electrode 222, the cathode electrode 222 can be formed as a light-transmitting transflective electrode. For example, the cathode electrode 222 is formed of a thin Ag film having a thickness of 10 nm so as to have light transmittance of approximately 60%. In this case, the photodiode PD can detect first light LD from a first surface FD side of the optical sensor 10, for example.

The protective film 23 is provided so as to cover the anode electrode 226. The protective film 23 is a passivation film provided to protect the photodiode PD.

While being worn on the object to be detected Fg, the detection device 100 turns on the light source 60 at the time of detection. The time of detection includes, for example, a preset date and time or a preset time, and a time when an instruction of detection has been given. The light emitted by the light source 60 that is turned on is received by the optical sensor 10 via the object to be detected Fg.

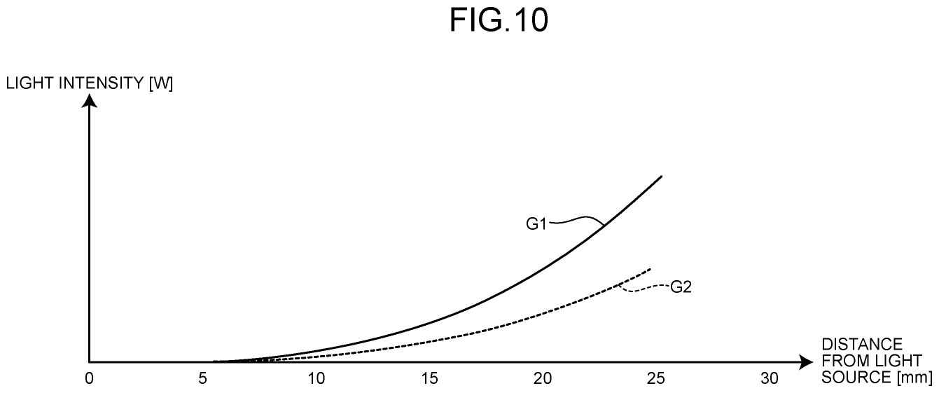

The following describes a measurement example by the optical sensor 10 of the detection device 100. FIG. 10 is a diagram illustrating exemplary relations between the distance DS and a light intensity of the optical sensor 10 illustrated in FIG. 4. In FIG. 10, the horizontal axis represents the distance DS (mm) at the optical sensor 10 from the light source 60, and the vertical axis represents the light intensity (W) corresponding to the distance DS. The light intensity indicates the intensity of the light emitted by the light source 60. A graph G1 illustrates a relation between the distance and the light intensity when the gate lines GCL are driven one by one and the measurements are sequentially performed. A graph G2 illustrates a relation between the distance and the light intensity when more than one of the gate lines GCL are driven at a time and the measurements are sequentially performed.

In the embodiment, in the detection device 100, as illustrated in FIG. 2, an imaginary line L1 connecting the light source 60 to a center 200C of the housing 200 is orthogonal to an imaginary line L2. The detection device 100 has characteristics that a region 300 from the imaginary line L1 to an imaginary line L3 connecting the center 200C of the housing 200 to the optical sensor 10 is roughly a portion where the sensitivity of the optical sensor 10 is the maximum when the housing 200 is worn on the object to be detected Fg.

The graphs G1 and G2 in FIG. 10 illustrate the respective light intensities measured by the optical sensor 10 at distances DS of 5.5 mm, 10.5 mm, 15.5 mm, 20.5 mm, and 25.5 mm from the light source 60 when the brightness (light intensity) of the light source 60 is constant. As illustrated in FIG. 10, in the detection device 100 of the embodiment, the planar optical sensor 10 cannot measure the light intensity when being close to the light source 60 because the brightness is too high. When the distance DS from the light source 60 is too long, the optical sensor 10 cannot measure the light intensity because of excessive darkness. Therefore, the detection device 100 of the present embodiment provides a technique to effectively use the entire detection area AA of the optical sensor 10 even when the planar optical sensor 10 and the light source 60 are arranged close to each other in the housing 200.

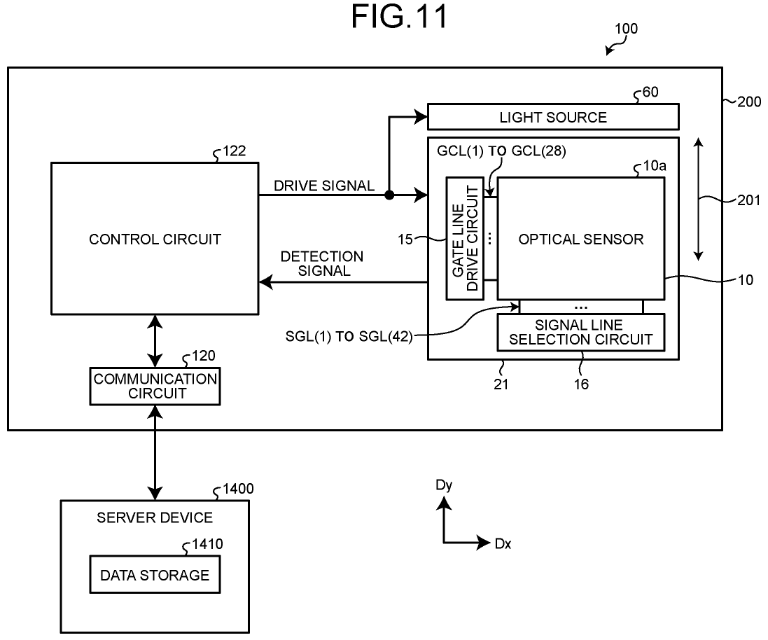

The following describes a functional configuration of the detection device 100 according to the embodiment. FIG. 11 is a diagram for explaining a control example of the control circuit 122. In the example illustrated in FIG. 11, a case will be described where the gate line drive circuit 15 includes 28 gate lines GCL(1), GCL(2), . . . , GCL(28), and the signal line selection circuit 16 includes 42 signal lines SGL(1), SGL(2), . . . , SGL(42). However, the number of the lines in each of the circuits is not limited to this case.

In the detection device 100, by supplying drive signals to the gate line drive circuit 15 and the light source 60, the control circuit 122 sequentially controls the lighting and non-lighting of the light source 60 and sequentially drives each of the gate lines GCL. The control circuit 122 stores, in the storage circuit 46 or the like, control information indicating a correspondence relation between each of the gate lines GCL(1), GCL(2), . . . , GCL(28) and the total amount of light of the light source 60 based on the relation between the light intensity and the distance DS from the light source 60 illustrated in FIG. 10. The total amount of light corresponds to the distance DS to the gate line GCL and is the product of the light intensity emitted by the light source 60 and the light-emitting time. The distance DS to the gate line GCL indicates a scan position in the optical sensor 10 of the detection device 100. The control information includes, for example, information indicating a one-to-one relation between the gate lines GCL(1), GCL(2), . . . , GCL(28) and the total amounts of light of the light source 60, and information indicating a relation between a gate line group including more than one of the gate lines GCL and the total amount of light of the light source 60. The control information includes, for example, a table allowing identification of a relation between, for example, an irradiation time of the light source 60, a value of current (hereinafter, referred to as a current value) applied corresponding to the total amount of light, or the like and the gate lines GCL.

The detection device 100 further includes a communication circuit 120. The communication circuit 120 wirelessly communicates. The communication circuit 120 supports wireless communication standards. The communication standards include, for example, the communication standards for cellular phones, such as 3G, 4G, and 5G, and the short-range wireless communication standards. The communication circuit 120 supplies received information to the control circuit 122. The communication circuit 120 transmits various types of information requested by the control circuit 122 to the destination.

The control circuit 122 of the detection device 100 is configured to be capable of accessing a server device 1400 in the cloud through the communication circuit 120. The server device 1400 is a network server device and includes a data storage 1410. The data storage 1410 can store therein information allowing identification of results of determination of biometric information D10 detected by the detection device 100. The server device 1400 can provide various types information, such as the control information stored in the data storage 1410, to the detection device 100.

### First Control of Detection Device

In first control, the detection device 100 performs control to drive the gate lines GCL one line by one line and vary the lighting time of the light source 60.

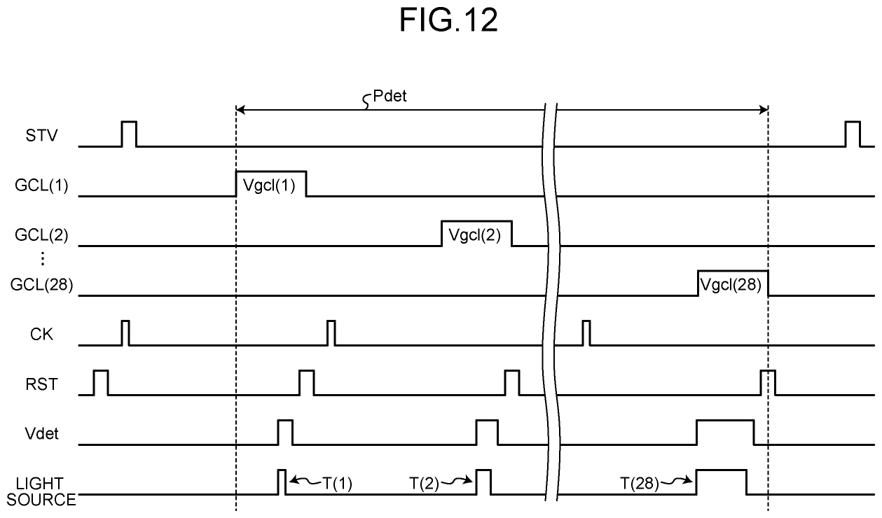

FIG. 12 is a timing waveform diagram illustrating an example of the first control of the detection device 100. As illustrated in FIG. 12, the gate line drive circuit 15 sequentially selects the gate lines GCL based on the start signal STV, the clock signal CK, and a reset signal RST. In a read period Pdet, the gate line drive circuit 15 supplies the gate drive signal Vgcl(1) at a high-level voltage (power supply voltage VDD) to the gate line GCL(1) based on a drive signal from the control circuit 122. Based on the control information, the control circuit 122 turns on the light source 60 by applying a predetermined voltage to the light source 60 for a time T(1) so as to achieve the total amount of light corresponding to the gate line GCL(1). After the light source 60 is turned off, the control circuit 122 sequentially supplies the selection signals ASW1, . . . , ASW6 to the signal line selection circuit 16 during a period in which the gate drive signal Vgcl(1) is at the high-level voltage (power supply voltage VDD). This operation sequentially or simultaneously couples the signal lines SGL of the partial detection areas PAA selected by the gate drive signal Vgcl(1) to the detection circuit 48. As a result, the control circuit 122 is coupled to the gate line GCL(1), and the detection signals Vdet of the photodiodes PD coupled to the signal lines SGL(1), SGL(2), . . . , SGL(42) are supplied to the detection circuit 48 for each of the partial detection areas PAA. The control circuit 122 can detect the light intensities (for example, peak values) of the photodiodes PD coupled to the gate line GCL(1) based on the signals from the detection circuit 48.

Subsequently, the gate line drive circuit 15 supplies the gate drive signal Vgcl(2) at the high-level voltage (power supply voltage VDD) to the gate line GCL(2) based on the clock signal CK and the reset signal RST. Based on the control information, the control circuit 122 turns on the light source 60 by applying the predetermined voltage to the light source 60 for a time T(2) so as to achieve the total amount of light corresponding to the gate line GCL(2). After the light source 60 is turned off, the control circuit 122 sequentially supplies the selection signals ASW1, . . . , ASW6 to the signal line selection circuit 16 during a period in which the gate drive signal Vgcl(2) is at the high-level voltage (power supply voltage VDD). This operation sequentially or simultaneously couples the signal lines SGL of the partial detection areas PAA selected by the gate drive signal Vgcl(2) to the detection circuit 48. As a result, the control circuit 122 is coupled to the gate line GCL(2), and the detection signals Vdet of the photodiodes PD coupled to the signal lines SGL(1), SGL(2), . . . , SGL(42) are supplied to the detection circuit 48 for each of the partial detection areas PAA. The control circuit 122 can detect the light intensities (for example, peak values) of the photodiodes PD coupled to the gate line GCL(2) based on the signals from the detection circuit 48. In this manner, the gate line drive circuit 15 performs the same operation for the other gate lines GCL(3) to GCL(27).

The gate line drive circuit 15 finally supplies the gate drive signal Vgcl(28) at the high-level voltage (power supply voltage VDD) to the gate line GCL(28) based on the clock signal CK and the reset signal RST. Based on the control information, the control circuit 122 turns on the light source 60 by applying the predetermined voltage to the light source 60 for a time T(28) so as to achieve the total amount of light corresponding to the gate line GCL(28). After the light source 60 is turned off, the control circuit 122 sequentially supplies the selection signals ASW1, . . . , ASW6 to the signal line selection circuit 16 during a period in which the gate drive signal Vgcl(28) is at the high-level voltage (power supply voltage VDD). This operation sequentially or simultaneously couples the signal lines SGL of the partial detection areas PAA selected by the gate drive signal Vgcl(28) to the detection circuit 48. As a result, the control circuit 122 is coupled to the gate line GCL(28), and the detection signals Vdet of the photodiodes PD coupled to the signal lines SGL(1), SGL(2), . . . , SGL(42) are supplied to the detection circuit 48 for each of the partial detection areas PAA. The control circuit 122 can detect the light intensities (for example, peak values) of the photodiodes PD coupled to the gate line GCL(28) based on the signals from the detection circuit 48.

As described above, in the detection device 100, the intensity of light itself emitted by the light source 60 is the same between when the gate line GCL(1) closest to the light source 60 is driven and when the gate line GCL(28) farthest from the light source 60 is driven. On the other hand, the lighting time of the light source 60 is varied according to the distance from the light source 60, thereby varying the total amount of light of the light source 60 when the light is detected by the photodiodes PD. This method allows the detection device 100 to cause the light from the light source 60 to reach the photodiodes PD at a light intensity suitable for the measurement range (characteristic) of the photodiodes PD with respect to the distance from the light source 60. As a result, by performing the first control, the detection device 100 can solve the conventional problem that the photodiodes PD near the light source 60 cannot make measurements because they are illuminated at excessively high brightness levels that exceed the measurement range and the photodiodes PD far from the light source 60 cannot make measurements because they are illuminated at excessively low brightness levels that do not reach the measurement range.

Thus, the detection device 100 can change the total amount of light reaching the optical sensor 10 from the light source 60 by changing the time of application of the voltage of the light source 60 for each of the gate lines GCL. Consequently, the total amount of light corresponding to the distance DS from the light source 60 can reach the optical sensor 10 even when the light source 60 and the one end 10a of the optical sensor 10 are located close to each other in the housing 200. As a result, the detection device 100 can detect the light in the entire detection area AA of the optical sensor 10 even when the planar optical sensor 10 and the light source 60 are arranged close to each other.

### Second Control of Detection Device

The following describes an example of second control of the detection device 100. In the second control, the detection device 100 performs control to drive more than one of the gate lines GCL at a time and vary the lighting time of the light source 60. In the present embodiment, a case will be described in which the detection device 100 drives two of the gate lines GCL at a time, but the number of the gate lines GCL that are driven at a time is not limited to this case. For example, the number of the gate lines GCL that are driven at a time can be set based on the relation between the distance DS and the light intensity of the optical sensor 10 described above.

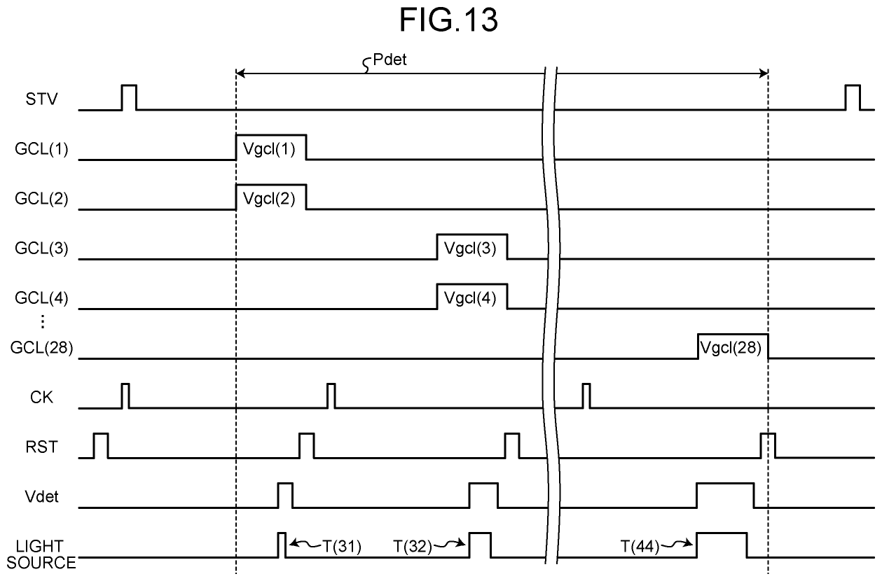

FIG. 13 is a timing waveform diagram illustrating an example of the second control of the detection device 100. As illustrated in FIG. 13, the gate line drive circuit 15 sequentially selects the gate lines GCL based on the start signal STV, the clock signal CK, and the reset signal RST. In the read period Pdet, the gate line drive circuit 15 supplies the gate drive signals Vgcl(1) and Vgcl(2) at the high-level voltage (power supply voltage VDD) to the gate lines GCL(1) and GCL(2) based on the drive signal from the control circuit 122. Based on the control information, the control circuit 122 turns on the light source 60 by applying the predetermined voltage to the light source 60 for a time T(31) so as to achieve the total amount of light corresponding to the gate lines GCL(1) and GCL(2). After the light source 60 is turned off, the control circuit 122 sequentially supplies the selection signals ASW1, . . . , ASW6 to the signal line selection circuit 16 during a period in which the gate drive signals Vgcl(1) and Vgcl(2) are at the high-level voltage (power supply voltage VDD). This operation sequentially or simultaneously couples the signal lines SGL of the partial detection areas PAA selected by the gate drive signals Vgcl(1) and Vgcl(2) to the detection circuit 48. As a result, the control circuit 122 is coupled to the gate lines GCL(1) and GCL(2), and the detection signals Vdet of the photodiodes PD coupled to the signal lines SGL(1), SGL(2), . . . , SGL(42) are supplied to the detection circuit 48 for each of the partial detection areas PAA. The control circuit 122 can detect the light intensities (for example, peak values) of the photodiodes PD coupled to the gate lines GCL(1) and GCL(2) based on the signals from the detection circuit 48.

Subsequently, the gate line drive circuit 15 supplies the gate drive signals Vgcl(3) and Vgcl(4) at the high-level voltage (power supply voltage VDD) to the gate lines GCL(3) and GCL(4) based on the clock signal CK and the reset signal RST. Based on the control information, the control circuit 122 turns on the light source 60 by applying the predetermined voltage to the light source 60 for a time T(32) so as to achieve the total amount of light corresponding to the gate lines GCL(3) and GCL(4). After the light source 60 is turned off, the control circuit 122 sequentially supplies the selection signals ASW1, . . . , ASW6 to the signal line selection circuit 16 during a period in which the gate drive signals Vgcl(3) and Vgcl(4) are at the high-level voltage (power supply voltage VDD). This operation sequentially or simultaneously couples the signal lines SGL of the partial detection areas PAA selected by the gate drive signals Vgcl(3) and Vgcl(4) to the detection circuit 48. As a result, the control circuit 122 is coupled to the gate lines GCL(3) and GCL(4), and the detection signals Vdet of the photodiodes PD coupled to the signal lines SGL(1), SGL(2), . . . , SGL(42) are supplied to the detection circuit 48 for each of the partial detection areas PAA. The control circuit 122 can detect the light intensities (for example, peak values) of the photodiodes PD coupled to the gate lines GCL(3) and GCL(4) based on the signals from the detection circuit 48. In this manner, the gate line drive circuit 15 performs the same operation for the gate lines GCL(5) to GCL(26).

The gate line drive circuit 15 finally supplies the gate drive signals Vgcl(27) and Vgcl(28) at the high-level voltage (power supply voltage VDD) to the gate lines GCL(27) and GCL(28) based on the clock signal CK and the reset signal RST. Based on the control information, the control circuit 122 turns on the light source 60 by applying the predetermined voltage to the light source 60 for a time T(44) so as to achieve the total amount of light corresponding to the gate lines GCL(27) and GCL(28). After the light source 60 is turned off, the control circuit 122 sequentially supplies the selection signals ASW1, . . . , ASW6 to the signal line selection circuit 16 during a period in which the gate drive signals Vgcl(27) and Vgcl(28) are at the high-level voltage (power supply voltage VDD). This operation sequentially or simultaneously couples the signal lines SGL of the partial detection areas PAA selected by the gate drive signals Vgcl(27) and Vgcl(28) to the detection circuit 48. As a result, the control circuit 122 is coupled to the gate lines GCL(27) and GCL(28), and the detection signals Vdet of the photodiodes PD coupled to the signal lines SGL(1), SGL(2), . . . , SGL(42) are supplied to the detection circuit 48 for each of the partial detection areas PAA. The control circuit 122 can detect the light intensities (for example, peak values) of the photodiodes PD coupled to the gate lines GCL(27) and GCL(28) based on the signals from the detection circuit 48.

As described above, in the detection device 100, the intensity of light itself emitted by the light source 60 is the same between when the gate line GCL(1) closest to the light source 60 and the gate line GCL(2) second closest to the light source 60 are driven and when the gate line GCL(28) farthest from the light source 60 and the gate line GCL(27) second farthest from the light source 60 are driven. On the other hand, the lighting time of the light source 60 is varied according to the distance from the light source 60, thereby varying the total amount of light of the light source 60 when the light is detected by the photodiodes PD. This method allows the detection device 100 to cause the light from the light source 60 to reach the photodiodes PD at a light intensity suitable for the measurement range (characteristic) of the photodiodes PD with respect to the distance from the light source 60. As a result, by performing the second control, the detection device 100 can solve the conventional problem that the photodiodes PD near the light source 60 cannot make measurements because they are illuminated at excessively high brightness levels that exceed the measurement range and the photodiodes PD far from the light source 60 cannot make measurements because they are illuminated at excessively low brightness levels that do not reach the measurement range.

Thus, the detection device 100 can change the total amount of light reaching the optical sensor 10 from the light source 60 by changing the time of application of the voltage of the light source 60 for each two gate lines GCL. Consequently, the total amount of light corresponding to the distance DS from the light source 60 can reach the optical sensor 10 even when the light source 60 and the one end 10a of the optical sensor 10 are located close to each other in the housing 200. As a result, the detection device 100 can detect the light in the entire detection area AA of the optical sensor 10 even when the planar optical sensor 10 and the light source 60 are arranged close to each other. Furthermore, the sensitivity of the optical sensor 10 is increased by controlling more than one of the gate lines GCL at a time. Therefore, the detection device 100 can perform the detection even when the light intensity is low and can reduce the measurement time.

### Third Control of Detection Device

The following describes an example of third control of the detection device 100. In the third control, the detection device 100 performs control to drive the gate lines GCL one line by one line and vary the value of current (current value) of the light source 60.

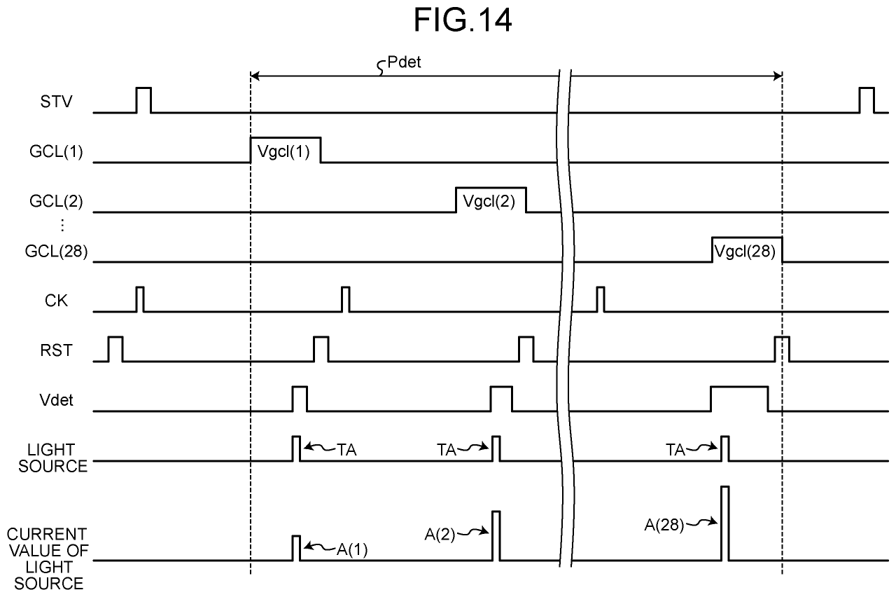

FIG. 14 is a timing waveform diagram illustrating an example of third control of the detection device 100. As illustrated in FIG. 14, the gate line drive circuit 15 sequentially selects the gate lines GCL based on the start signal STV, the clock signal CK, and the reset signal RST. In the read period Pdet, the gate line drive circuit 15 supplies the gate drive signal Vgcl(1) at the high-level voltage (power supply voltage VDD) to the gate line GCL(1) based on the drive signal from the control circuit 122. Based on the control information, the control circuit 122 turns on the light source 60 by applying a current of a current value A(1) to the light source 60 for a time TA so as to achieve the total amount of light corresponding to the gate line GCL(1). The current value A(1) and time TA are set to values corresponding to the distance DS from the light source 60 to the gate line GCL(1) and the light intensity. After the light source 60 is turned off, the control circuit 122 sequentially supplies the selection signals ASW1, . . . , ASW6 to the signal line selection circuit 16 during a period in which the gate drive signal Vgcl(1) is at the high-level voltage (power supply voltage VDD). This operation sequentially or simultaneously couples the signal lines SGL of the partial detection areas PAA selected by the gate drive signal Vgcl(1) to the detection circuit 48. As a result, the control circuit 122 is coupled to the gate line GCL(1), and the detection signals Vdet of the photodiodes PD coupled to the signal lines SGL(1), SGL(2), . . . , SGL(42) are supplied to the detection circuit 48 for each of the partial detection areas PAA. The control circuit 122 can detect the light intensities (for example, peak values) of the photodiodes PD coupled to the gate line GCL(1) based on the signals from the detection circuit 48.

Subsequently, the gate line drive circuit 15 supplies the gate drive signal Vgcl(2) at the high-level voltage (power supply voltage VDD) to the gate line GCL(2) based on the clock signal CK and the reset signal RST. Based on the control information, the control circuit 122 turns on the light source 60 by applying a current of a current value A(2) to the light source 60 for the same time TA as that mentioned above. The current value A(2) and the time TA are set to values corresponding to the distance DS from the light source 60 to the gate line GCL(2) and the light intensity. In the present embodiment, the current value A(2) is larger than the current value A(1). After the light source 60 is turned off, the control circuit 122 sequentially supplies the selection signals ASW1, . . . , ASW6 to the signal line selection circuit 16 during a period in which the gate drive signal Vgcl(2) is at the high-level voltage (power supply voltage VDD). This operation sequentially or simultaneously couples the signal lines SGL of the partial detection areas PAA selected by the gate drive signal Vgcl(2) to the detection circuit 48. As a result, the control circuit 122 is coupled to the gate line GCL(2), and the detection signals Vdet of the photodiodes PD coupled to the signal lines SGL(1), SGL(2), . . . , SGL(42) are supplied to the detection circuit 48 for each of the partial detection areas PAA. The control circuit 122 can detect the light intensities (for example, peak values) of the photodiodes PD coupled to the gate line GCL(2) based on the signals from the detection circuit 48. In this manner, the gate line drive circuit 15 performs the same operation for the other gate lines GCL(3) to GCL(27).

The gate line drive circuit 15 finally supplies the gate drive signal Vgcl(28) at the high-level voltage (power supply voltage VDD) to the gate line GCL(28) based on the clock signal CK and the reset signal RST. Based on the control information, the control circuit 122 turns on the light source 60 by applying a current of a current value A(28) to the light source 60 for the same time TA as that mentioned above so as to achieve the total amount of light corresponding to the gate line GCL(28). The current value A(28) and the time TA are set to values corresponding to the distance DS from the light source 60 to the gate line GCL(28) and the light intensity. In the present embodiment, the current value A(28) is larger than the current value A(2). After the light source 60 is turned off, the control circuit 122 sequentially supplies the selection signals ASW1, . . . , ASW6 to the signal line selection circuit 16 during a period in which the gate drive signal Vgcl(28) is at the high-level voltage (power supply voltage VDD). This operation sequentially or simultaneously couples the signal lines SGL of the partial detection areas PAA selected by the gate drive signal Vgcl(28) to the detection circuit 48. As a result, the control circuit 122 is coupled to the gate line GCL(28), and the detection signals Vdet of the photodiodes PD coupled to the signal lines SGL(1), SGL(2), . . . , SGL(42) are supplied to the detection circuit 48 for each of the partial detection areas PAA. The control circuit 122 can detect the light intensities (for example, peak values) of the photodiodes PD coupled to the gate line GCL(28) based on the signals from the detection circuit 48.

As described above, in the detection device 100, the light-emitting time itself of the light source 60 is the same between when the gate line GCL(1) closest to the light source 60 is driven and when the gate line GCL(28) farthest from the light source 60 is driven. On the other hand, the current value of the light source 60 is varied according to the distance from the light source 60, thereby varying the total amount of light of the light source 60 when the light is detected by the photodiodes PD. This method allows the detection device 100 to cause the light from the light source 60 to reach the photodiodes PD at a light intensity suitable for the measurement range (characteristic) of the photodiodes PD that varies depending on the distance from the light source 60. As a result, by performing the third control, the detection device 100 can solve the conventional problem that the photodiodes PD near the light source 60 cannot make measurements because they are illuminated at excessively high brightness levels that exceed the measurement range and the photodiodes PD far from the light source 60 cannot make measurements because they are illuminated at excessively low brightness levels that do not reach the measurement range.

Thus, the detection device 100 can change the total amount of light reaching the optical sensor 10 from the light source 60 by changing the current value of the light source 60 for each of the gate lines GCL. Consequently, the total amount of light corresponding to the distance DS from the light source 60 can reach the optical sensor 10 even when the light source 60 and the one end 10a of the optical sensor 10 are located close to each other in the housing 200. As a result, the detection device 100 can detect the light in the entire detection area AA of the optical sensor 10 even when the planar optical sensor 10 and the light source 60 are arranged close to each other. Furthermore, the detection device 100 can reduce the measurement time by changing the current value of the light source 60.

### Fourth Control of Detection Device

The following describes an example of fourth control of the detection device 100. In the fourth control, the detection device 100 performs control to drive more than one of the gate lines GCL that are driven at a time in the same manner as in the second control described above and vary the current value of the light source 60 in the same manner as in the third control described above.

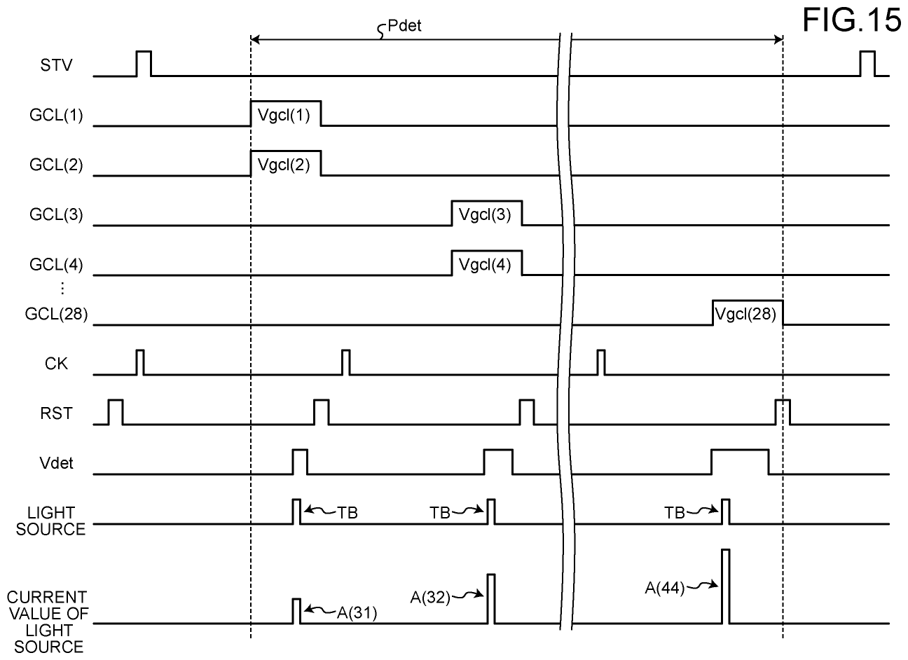

FIG. 15 is a timing waveform diagram illustrating an example of the fourth control of the detection device 100. As illustrated in FIG. 15, the gate line drive circuit 15 sequentially selects the gate lines GCL based on the start signal STV, the clock signal CK, and the reset signal RST. In the read period Pdet, the gate line drive circuit 15 supplies the gate drive signals Vgcl(1) and Vgcl(2) at the high-level voltage (power supply voltage VDD) to the gate lines GCL(1) and GCL(2) based on the drive signal from the control circuit 122. Based on the control information, the control circuit 122 turns on the light source 60 by applying a current of a current value A(31) to the light source 60 for a time TB so as to achieve the total amount of light corresponding to the gate lines GCL(1) and GCL(2). The current value A(31) and the time TB are set to values corresponding to the distances DS from the light source 60 to the gate lines GCL(1) and GCL(2) and the light intensity. That is, the product of a current value A(31) and the time TB is the total amount of light to the gate lines GCL(1) and GCL(2). After the light source 60 is turned off, the control circuit 122 sequentially supplies the selection signals ASW1, . . . , ASW6 to the signal line selection circuit 16 during a period in which the gate drive signals Vgcl(1) and Vgcl(2) are at the high-level voltage (power supply voltage VDD). This operation sequentially or simultaneously couples the signal lines SGL of the partial detection areas PAA selected by the gate drive signals Vgcl(1) and Vgcl(2) to the detection circuit 48. As a result, the control circuit 122 is coupled to the gate lines GCL(1) and GCL(2), and the detection signals Vdet of the photodiodes PD coupled to the signal lines SGL(1), SGL(2), . . . , SGL(42) are supplied to the detection circuit 48 for each of the partial detection areas PAA. The control circuit 122 can detect the light intensities (for example, peak values) of the photodiodes PD coupled to the gate lines GCL(1) and GCL(2) based on the signals from the detection circuit 48.

Subsequently, the gate line drive circuit 15 supplies the gate drive signals Vgcl(3) and Vgcl(4) at the high-level voltage (power supply voltage VDD) to the gate lines GCL(3) and GCL(4) based on the clock signal CK and the reset signal RST. Based on the control information, the control circuit 122 turns on the light source 60 by applying a current of a current value A(32) to the light source 60 for the same time TB as that mentioned above. The current value A(32) and the time TB are set to values corresponding to the distances DS from the light source 60 to the gate lines GCL(3) and GCL(4) and the light intensity. That is, the product of the current value A(32) and the time TB is the total amount of light to the gate lines GCL(3) and GCL(4). After the light source 60 is turned off, the control circuit 122 sequentially supplies the selection signals ASW1, . . . , ASW6 to the signal line selection circuit 16 during a period in which the gate drive signals Vgcl(3) and Vgcl(4) are at the high-level voltage (power supply voltage VDD). This operation sequentially or simultaneously couples the signal lines SGL of the partial detection areas PAA selected by the gate drive signals Vgcl(3) and Vgcl(4) to the detection circuit 48. As a result, the control circuit 122 is coupled to the gate lines GCL(3) and GCL(4), and the detection signals Vdet of the photodiodes PD coupled to the signal lines SGL(1), SGL(2), . . . , SGL(42) are supplied to the detection circuit 48 for each of the partial detection areas PAA. The control circuit 122 can detect the light intensities (for example, peak values) of the photodiodes PD coupled to the gate lines GCL(3) and GCL(4) based on the signals from the detection circuit 48. In this manner, the gate line drive circuit 15 performs the same operation for the gate lines GCL(5) to GCL(26).

The gate line drive circuit 15 finally supplies the gate drive signals Vgcl(27) and Vgcl(28) at the high-level voltage (power supply voltage VDD) to the gate lines GCL(27) and GCL(28) based on the clock signal CK and the reset signal RST. Based on the control information, the control circuit 122 turns on the light source 60 by applying a current of a current value A(44) to the light source 60 for the same time TB as that mentioned above so as to achieve the total amount of light corresponding to the gate lines GCL(27) and GCL(28). The current value A(44) and the time TB are set to values corresponding to the distances DS from the light source 60 to the gate lines GCL(27) and GCL(28) and the light intensity. That is, the product of the current value A(44) and the time TB is the total amount of light to the gate lines GCL(27) and GCL(28). After the light source 60 is turned off, the control circuit 122 sequentially supplies the selection signals ASW1, . . . , ASW6 to the signal line selection circuit 16 during a period in which the gate drive signals Vgcl(27) and Vgcl(28) are at the high-level voltage (power supply voltage VDD). This operation sequentially or simultaneously couples the signal lines SGL of the partial detection areas PAA selected by the gate drive signals Vgcl(27) and Vgcl(28) to the detection circuit 48. As a result, the control circuit 122 is coupled to the gate lines GCL(27) and GCL(28), and the detection signals Vdet of the photodiodes PD coupled to the signal lines SGL(1), SGL(2), . . . , SGL(42) are supplied to the detection circuit 48 for each of the partial detection areas PAA. The control circuit 122 can detect the light intensities (for example, peak values) of the photodiodes PD coupled to the gate lines GCL(27) and GCL(28) based on the signals from the detection circuit 48.

As described above, in the detection device 100, the light-emitting time itself of the light source 60 is the same between when the gate line GCL(1) closest to the light source 60 and the gate line GCL(2) second closest to the light source 60 are driven and when the gate line GCL(28) farthest from the light source 60 and the gate line GCL(27) second farthest from the light source 60 are driven. On the other hand, the current value of the light source 60 is varied according to the distance from the light source 60, thereby varying the total amount of light of the light source 60 when the light is detected by the photodiodes PD. This method allows the detection device 100 to cause the light from the light source 60 to reach the photodiodes PD at a light intensity suitable for the measurement range (characteristic) of the photodiodes PD that varies depending on the distance from the light source 60. As a result, by performing the fourth control, the detection device 100 can solve the conventional problem that the photodiodes PD near the light source 60 cannot make measurements because they are illuminated at excessively high brightness levels that exceed the measurement range and the photodiodes PD far from the light source 60 are cannot make measurements because they are illuminated at excessively low brightness levels that do not reach the measurement range.

Thus, the detection device 100 can change the total amount of light reaching the optical sensor 10 from the light source 60 by changing the current value of the light source 60 for each two gate lines GCL. Consequently, the total amount of light corresponding to the distance DS from the light source 60 can reach the optical sensor 10 even when the light source 60 and the one end 10a of the optical sensor 10 are located close to each other in the housing 200. As a result, the detection device 100 can detect the light in the entire detection area AA of the optical sensor 10 even when the planar optical sensor 10 and the light source 60 are arranged close to each other. Furthermore, the sensitivity of the optical sensor 10 is increased by controlling more than one of the gate lines GCL at a time. Therefore, the detection device 100 can perform the detection even when the light intensity is low and can reduce the measurement time.

The detection device 100 described above can perform the accurate detection by disposing the one end 10a of the optical sensor 10 including the organic photodiodes (OPDs) having good light-receiving sensitivity close to the light source 60. This configuration allows the detection device 100 to accurately detect the light over the entire detection area AA of the optical sensor 10 even when the planar optical sensor 10 and the light source 60 are arranged in the housing 200.

### Other Embodiments

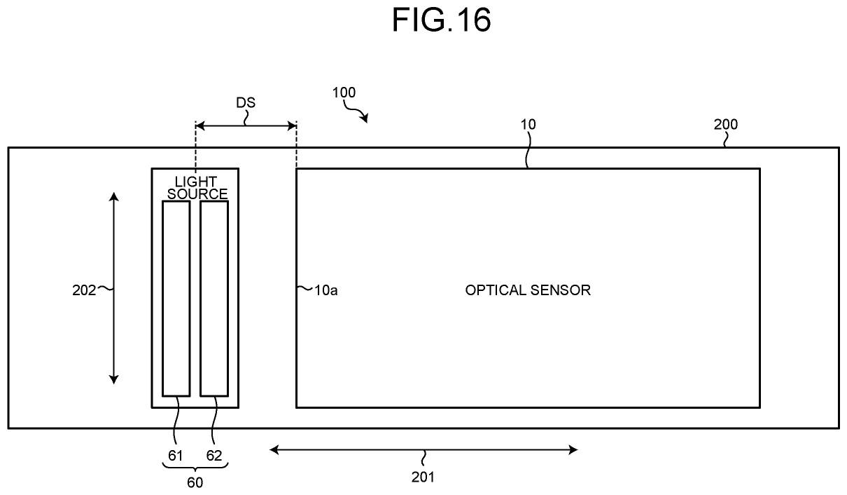

FIG. 16 is a schematic view obtained by developing another configuration example of the light source of the detection device 100 according to the embodiment. As illustrated in FIG. 16, the detection device 100 may have a configuration including a light source 60 that includes the first light source 61 and the second light source 62 arranged along the peripheral direction 201. The first light source 61 includes, for example, an LED that emits infrared light. The second light source 62 includes, for example, an LED that emits red light. The first and the second light sources 61 and 62 may each be made of, for example, one LED or a plurality of LEDs. The detection device 100 may have a configuration including three or more light sources. In the present embodiment, the distance DS is the distance from a position between the first and the second light sources 61 and 62 to the optical sensor 10, but can be the distance from any position of the light source 60 to the optical sensor 10. The distance DS may be, for example, the respective distances from the first and the second light sources 61 and 62, and the control information described above may be information indicating a relation between the total amount of light of the light sources and the gate line GCL.

The detection device 100 uses the optical sensor 10 to detect waveform data for each of the gate lines GCL with the first light source 61 turned on, and then uses the optical sensor 10 to detect the waveform data for each of the gate lines GCL with the first light source 61 turned off and the second light source 62 turned on. In this case, the control circuit 122 of the detection device 100 turns on the first light source 61 or the second light source 62 so as to achieve the total amount of light corresponding to the gate line GCL of the photodiodes PD exposed to the light of the light source 60. Thus, the detection device 100 alternately turns on the first and the second light sources 61 and 62 and acquires signals having respective wavelength components thereof in a time-division manner. The detection device 100 then performs processing to calculate the blood oxygen saturation level (SpO₂) from the ratio of hemoglobin absorbance between two wavelengths (red light/infrared light). The detection device 100 displays the biometric information including the blood oxygen saturation level and the like on a display device and/or transmits it through the communication circuit 120.

Thus, the detection device 100 can detect the light from the light source 60 using the optical sensor 10 by alternately switching on the first and the second light sources 61 and 62 and exposing the photodiodes PD with the light therefrom for each of the gate lines GCL. Consequently, the total amount of light corresponding to the distance DS from the light source 60 can reach the optical sensor 10 even when a plurality of the light sources 60 and the one end 10a of the optical sensor 10 are located close to each other in the housing 200. As a result, the detection device 100 can detect the light in the entire detection area AA of the optical sensor 10 even when the planar optical sensor 10 and the light source 60 are arranged close to each other.

While the case has been described where the housing 200 of the detection device 100 described above has a ring shape, the shape of the housing 200 is not limited to this case. Even if the housing 200 is, for example, square, flat-plate shaped, or sheet-shaped, the detection device 100 can detect the light in the entire detection area AA of the optical sensor 10 with the planar optical sensor 10 and the light source 60 arranged close to each other.

The components in the embodiments described above can be combined as appropriate. Other operational advantages accruing from the aspects described in the embodiments of the present disclosure that are obvious from the description herein, or that are conceivable as appropriate by those skilled in the art will naturally be understood as accruing from the present disclosure.

What is claimed is:

1. A detection device comprising:
a housing;
an optical sensor that comprises a plurality of photodiodes provided in a matrix having a row-column configuration and is provided in a planar configuration in the housing;
a light source disposed in the housing so as to be close to an end of the optical sensor; and
a control circuit configured to control light emission of the light source, wherein
the optical sensor comprises a plurality of gate lines arranged in a first direction away from the light source and electrically coupled to the photodiodes, and
the control circuit is configured to control the light emission of the light source such that, when the photodiodes are sequentially driven on a gate line basis, a total amount of light of the light source when detected by the photodiodes driven by the gate line closest to the light source is smaller than the total amount of light of the light source when detected by the photodiodes driven by the gate line farthest from the light source.

2. The detection device according to claim 1, wherein the control circuit is configured to drive more than one of the gate lines at a time and control the light emission of the light source so as to vary the total amount of light for each driving operation.

3. The detection device according to claim 2, wherein the control circuit is configured to vary the total amount of light emitted by the light source by varying a lighting time of the light source.

4. The detection device according to claim 3, wherein
the light source is capable of emitting light having a plurality of different wavelengths, and
the control circuit is configured to vary the total amount of light of the light source for each of the wavelengths.

5. The detection device according to claim 4, wherein each of the photodiodes comprises a lower electrode, a lower buffer layer, an active layer, an upper buffer layer, and an upper electrode that are stacked on a substrate in the order as listed.

6. The detection device according to claim 5, wherein
the housing is formed in a ring shape, and
the light source and the optical sensor are adjacently arranged in a peripheral direction of the housing.

7. The detection device according to claim 2, wherein the control circuit is configured to vary the total amount of light emitted by the light source by varying a value of current flowing through the light source.

8. The detection device according to claim 7, wherein
the light source is capable of emitting light having a plurality of different wavelengths, and
the control circuit is configured to vary the total amount of light of the light source for each of the wavelengths.

9. The detection device according to claim 8, wherein each of the photodiodes comprises a lower electrode, a lower buffer layer, an active layer, an upper buffer layer, and an upper electrode that are stacked on a substrate in the order as listed.

10. The detection device according to claim 9, wherein
the housing is formed in a ring shape, and
the light source and the optical sensor are adjacently arranged in a peripheral direction of the housing.
